(19) United States
(12) Patent Application Publication
(10) Pub. No.: US 2025/0082516 A1
(43) Pub. Date: Mar. 13, 2025

(54) CATAMENIAL PROTECTION DEVICE AND METHOD FOR PRODUCING THE SAME

(71) Applicant: Sibship AB, Östersund (SE)

(72) Inventor: Louise BERG, Östersund (SE)

(73) Assignee: Sibship AB, Östersund (SE)

(21) Appl. No.: 18/863,961

(22) PCT Filed: May 9, 2023
(86) PCT No.: PCT/SE2023/050451

§ 371 Date: Nov. 7, 2024

(30) Foreign Application Priority Data
May 9, 2022 (SE) 2250561-4
May 9, 2022 (SE) 2250562-2
May 9, 2022 (SE) 2250563-0

(51) Int. Cl. A61F 13/20

(52) U.S. Cl. CPC A61F 13/202

(57) ABSTRACT

The disclosure relates to a catamenial protection device for absorbing menstrual fluid when positioned inside a vagina. A shell is arranged to enclose an inner body of the catamenial protection device. The shelf includes at least one elongated portion arranged such that the shell is enabled to expand or retract.

### TECHNICAL FIELD

The present application relates in general to menstrual protection, and especially to a catamenial protection device for absorbing menstrual fluid when positioned inside a vagina. The catamenial protection device comprises an inner body comprising material with high fluid absorbing properties and a shell provided with a hard outer surface and enclosing the inner body.

### BACKGROUND

A commonly used catamenial protection hygiene product for women are tampons, which are inserted into the vagina of the user and absorbs menstrual fluids within the vagina. Tampons has historically comprised an absorbent core containing superabsorbent materials enclosed by a fluid-permeable wrapper. A general problem with these type of hygiene products is that the sensitive mucous member of the vagina is irritated by the tampon, partly by that the contact per se may chafe and irritate, and partly by the risk of growth of bacteria causing different types of complications such as Toxic shock syndrome, TSS. TSS is a sudden and potentially fatal condition. It is caused by the release of toxins from an overgrowth of bacteria called Staphylococcus aureus, or staph, which is found in many women's bodies.

Toxic shock syndrome affects menstruating women, especially those who use super-absorbent tampons that is in use for in this context relatively long term. The growth of bacteria is accelerated by the combination of old menstrual fluid being in contact with the mucous member and long term use of tampons.

If TSS occurs, the affected will become very acutely ill with fever and rash, which is almost like a tan. They often become strongly red in the eyes as well as in the oral mucosa and swell up in the palms and soles of the feet. Many vomit and have diarrhea. The most seriously ill patients can develop a shock image. Laboratory tests often show effects on the kidneys, liver and blood, among other things. If the patient does not come for treatment, the mortality rate is high. The disease is treated with antibiotics and, if necessary, drugs for shock.

For exposed women, with hard working conditions with few opportunities to breaks during work this may be a serious problem, especially since these women often have limited access to medical care and medications. For these women a proper female hygiene during menstrual periods may be difficult to achieve, increasing the risk of complications like TSS. Therefore, there is a need of an improved catamenial protection device, at least minimizing these type of problems.

Another problem with traditional menstrual protections devices is that they are often made of materials which need to be sorted out as combustible materials in the waste bin. Thus, there is a need to reduce the amount of waste created in connection with the use of those products.

### SUMMARY

An object of the present disclosure is to provide an improved catamenial protection device which addressed at least some of the problems related with present products. A further object is to provide a method for producing a catamenial protection device.

In a first aspect, a catamenial protection device for absorbing menstrual fluid when positioned inside a vagina is provided. The catamenial protection device may comprise an inner body comprising material with high fluid absorbing properties and a shell provided with a hard outer surface and enclosing the inner body. The shell may comprise at least one elongated portion arranged such that two long sides of the at least one elongated portion are enabled to at least partly overlap each other, and such that one or more pathways for transporting menstrual fluid may be provided in the overlapping area of the overlapping long sides.

In embodiments of the catamenial protection device, the overlapping long sides may be arranged at least partly at a distance from each other. Further, the pathways may be arranged to enable transport of menstrual fluid in a substantially transversal direction in relation to a longitudinal axis may be of the catamenial protection device when the device is in use. Still further, at least one of the long sides may be provided with at least one protruding part, wherein the at least one protruding part may be arranged to be in contact with the overlapping long side, and whereby the one or more paths ways for transporting menstrual fluid may be provided on one or both sides of the at least one protruding part.

In other embodiments of the catamenial protection device, the at least one protruding part may have a substantially circular shape. The at least one protruding part may alternatively have a substantially elongated shape extending in a substantially transversal direction with an inclined angle a in relation to the direction of an axis. In other embodiments herein, at least one of the overlapping long sides may be provided with a plurality of protruding parts whereby the pathways may be provided between two adjacent protruding parts. Alternatively, both of the overlapping long sides may be provided with one or more protruding parts whereby the pathways may be provided between two adjacent protruding parts. The one or more protruding parts may be harder than the surrounding surface of the elongated portions.

In yet other embodiments of the catamenial protection device, the shell may comprise a plurality of elongated portions and the long sides enabled to at least partly overlap each other are a respective long side of two adjacent elongated portions. The shell may in a first end further comprise a bottom arranged to connect to the elongated portion or portions and each of the elongated portions may be provided with a shape enabling the shell to be folded together. The plurality of elongated portions may be connected to each other in a first end, and may be arranged to enable an opening at a second end, wherein the second end is facing towards a proximal portion of the vagina when the device is in use.

In other embodiments of the catamenial protection device, the one or more pathways may be provided with a hard surface whereby transport of menstrual fluid is facilitated. The length of the one or more pathways in the substantially transversal direction may be longer than the thickness of the walls of the shell.

In embodiments of the catamenial protection device, the at least one elongated portion may be arranged such that the overlapping long sides are enabled to slide in relation to each other, whereby the device is enabled to expand or retract.

In embodiments of the catamenial protection device, the shell may comprise material with low fluid absorbing properties. The shell and/or the inner body may comprise environmentally friendly material. The environmentally friendly material may be biodegradable and/or compostable. The biodegradable material may be biodegradable cellulose made from a mix of long fiber kraft, such as NBSK, and short fiber, such as BCTMP. The environmentally friendly material may alternatively be biodegradable plastics, or the biodegradable material may be substantially pulp slurry. The pulp or pulp slurry may comprise cellulose, hemicellulose and lignin. The lignin may be evenly distributed on the outer surface of the shell whereby a smooth and hard outer surface is provided.

In other embodiments of the catamenial protection device, the at least one elongated portion may be provided with a substantially concave shape. The shell may be provided with a rough inner surface. In still other embodiments of the catamenial protection device, the device may further comprise an intermediate pouch enclosing the inner body.

In embodiments of a catamenial protection device, the shell (100) may have a thickness of 0.05-2.0 mm. The shell (100) may have a thickness of between 0.1-1.0 mm. The shell (100) may have a maximal liquid absorption capacity of 0.2-2.0 g H2O per g weight. The shell (100) may have a maximal liquid absorption of 0.5 g H2O per g weight.

According to other aspects, a method of producing a catamenial protection device is provided. The method comprises molding a shell by applying a pulp or pulp slurry in a first mold half provided with a molding surface with at least one cavity with a shape corresponding to at least one elongated portion, pressing the applied pulp or pulp slurry between the first mold half and a mating second mold half, heating the molded shell at an elevated temperature and under pressure, and arranging an inner body in the shell.

In alternative embodiments, methods herein of producing a catamenial protection device may further comprise folding the molded shell together whereby the shell at least partly encloses the inner body. One cavity may be provided with at least one recess with a shape corresponding to at least one protruding part of the shell and pressing may be performed such that the slurry is pressed into the at least one recess. Heating may be performed by heating the first mold half, whereby the heat distribution in the slurry during heating will be warmer closest to the first mold half, and colder closest to the second mold half. The molding surface may be provided with a plurality of cavities with a shape corresponding to a plurality of elongated portions and an intermediate part providing for molding a bottom in a first end the bottom is connected to the one or more elongated portion, and folding the shell may be performed by folding the one or more elongated portion together.

According to yet other aspects, a shell for use in a catamenial protection device for absorbing menstrual fluid when positioned inside a vagina is provided. The shell is arranged to enclose an inner body of the catamenial protection device, and the shell may comprise at least one elongated portion arranged such that the shell is enabled to expand or retract.

In embodiments of a shell for use in a catamenial protection device, the at least one elongated portion may be arranged such that two long sides of the at least one elongated portion are enabled to at least partly overlap each other, and one or more pathways for transporting menstrual fluid may be provided in the overlapping area of the overlapping long sides. The overlapping long sides may be arranged at least partly at a distance from each other. The shell may be arranged to be folded together whereby the inner body is enclosed by the shell.

In embodiments of a shell for use in a catamenial protection device, the end portion of the at least one elongated portion may be folded towards the center of the shell. The shell when folded together may be provided with a shape cooperating with the shape of the inner body. The shell may further be provided with a hard outer surface. The pathways may be arranged to enable transport of menstrual fluid in a substantially transversal direction in relation to a longitudinal axis may be of the catamenial protection device when the device is in use. At least one of the long sides may be provided with at least one protruding part and the at least one protruding part may be arranged to be in contact with the overlapping long side, whereby the one or more pathways for transporting menstrual fluid may be provided on one or both sides of the at least one protruding part.

In embodiments of a shell for use in a catamenial protection device, the shell may comprise a plurality of elongated portions and the long sides enabled to at least partly overlap each other are a respective long side of two adjacent elongated portions. The shell may in a first end further comprise a bottom arranged to connect the plurality of elongated portions to each other, and each of the plurality of elongated portions may be provided with a shape enable them to be folded together towards a second end. The shell when folded together may be provided with a shape cooperating with the shape of the inner body.

In embodiments of a shell for use in a catamenial protection device, the at least one protruding part may have a substantially circular shape. The at least one protruding part may alternatively have a substantially elongated shape and may extend in a substantially transversal direction with an inclined angle a in relation to the direction of the axis.

In embodiments of a shell for use in a catamenial protection device, at least one of the overlapping long sides may be provided with a plurality of protruding parts whereby the pathways may be provided between two adjacent protruding parts. The pathways may be provided with a hard surface whereby transport of menstrual fluid is facilitated. The length of the pathways in the substantially transversal direction may be longer than the thickness of the walls of the shell.

In embodiments of a shell for use in a catamenial protection device, the plurality of elongated portions may be connected to each other in a first end, and may be arranged to enable an opening at a second end. The second end is facing towards a proximal portion of the vagina when the device is in use. The at least one elongated portion may be arranged such that the overlapping long sides are enabled to slide in relation to each other. The one or more protruding parts may be harder than the surrounding surface of the elongated portions.

In embodiments of a shell for use in a catamenial protection device, the shell may comprise material with low fluid absorbing properties. The shell may comprise environmentally friendly material. The environmentally friendly material may be biodegradable. The biodegradable material may be biodegradable cellulose made from a mix of long fiber kraft, such as NBSK, and short fiber, such as BCTMP. The environmentally friendly material may be biodegradable plastics. The biodegradable material may be based on pulp or pulp slurry. The pulp or pulp slurry may comprise cellulose, hemicellulose and lignin. The lignin may be evenly distributed on the outer surface of the shell whereby a smooth and hard outer surface is provided.

In embodiments of a shell for use in a catamenial protection device, the at least one elongated portion may be provided with a substantially concave shape.

In embodiments of a shell for use in a catamenial protection device, the shell is provided with a rough inner surface.

In embodiments of a shell for use in a catamenial protection device, the shell (100) may have a thickness of 0.05-2.0 mm. The shell (100) may have a thickness of between 0.1-1.0 mm. The shell (100) may have a maximal liquid absorption capacity of 0.2-2.0 g H2O per g weight. The shell (100) may have a maximal liquid absorption of 0.5 g H2O per g weight.

In another aspect, a method of producing a shell is provided. The method comprises molding a shell by applying a pulp or pulp slurry in a first mold half provided with a molding surface with at least one cavity with a shape corresponding to at least one elongated portion pressing the applied pulp or pulp slurry between the first mold half and a mating second mold half, heating the molded shell at an elevated temperature and under pressure.

In embodiments, methods herein of producing a shell may further comprise folding the molded shell together whereby the shell is enabled to at least partly enclose an inner body of a catamenial protection device. The at least one cavity may be provided with at least one recess with a shape corresponding to at least one protruding part of the shell and pressing may be performed such that the slurry is pressed into the at least one recess. Heating may be performed by heating the first mold half, whereby the heat distribution in the slurry during heating will be warmer closest to the first mold half, and colder closest to the second mold half.

In embodiments, the molding surface may be provided with a plurality of cavities with a shape corresponding to a plurality of elongated portions and an intermediate part providing for molding a bottom. In a first end, the bottom may connect the plurality of elongated portions to each other, and folding the shell may be performed by folding the plurality of elongated portions together towards a second end.

In yet another aspect, a shell for use in a catamenial protection device for absorbing menstrual fluid when positioned inside a vagina is provided. The shell may be arranged to enclose an inner body of the catamenial protection device. The shell is provided with a hard outer surface and the shell is provided with a rough inner surface.

In embodiments, the shell may comprise material with low fluid absorbing properties. The shell may comprise environmentally friendly material. The environmentally friendly material may be biodegradable. The biodegradable material may be biodegradable cellulose made from a mix of long fiber kraft, such as NBSK, and short fiber, such as BCTMP. The environmentally friendly material may be biodegradable plastics. The biodegradable material may alternatively be based on pulp slurry. The pulp or pulp slurry may comprise cellulose, hemicellulose and lignin. The lignin may be evenly distributed on the outer surface of the shell whereby a smooth and hard outer surface is provided.

In embodiments, pathways may be arranged to enable transport of menstrual fluid in a substantially transversal direction in relation to a longitudinal axis may be of the catamenial protection device when the device is in use. The pathways may be provided with a hard surface whereby transport of menstrual fluid is facilitated.

### BRIEF DESCRIPTION

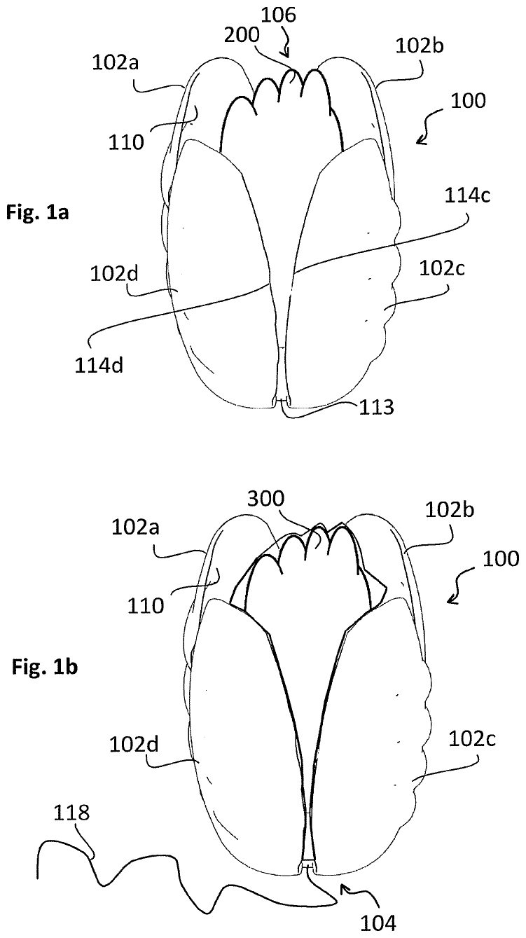

FIG. 1a and b show a catamenial protection device in accordance with embodiments herein.

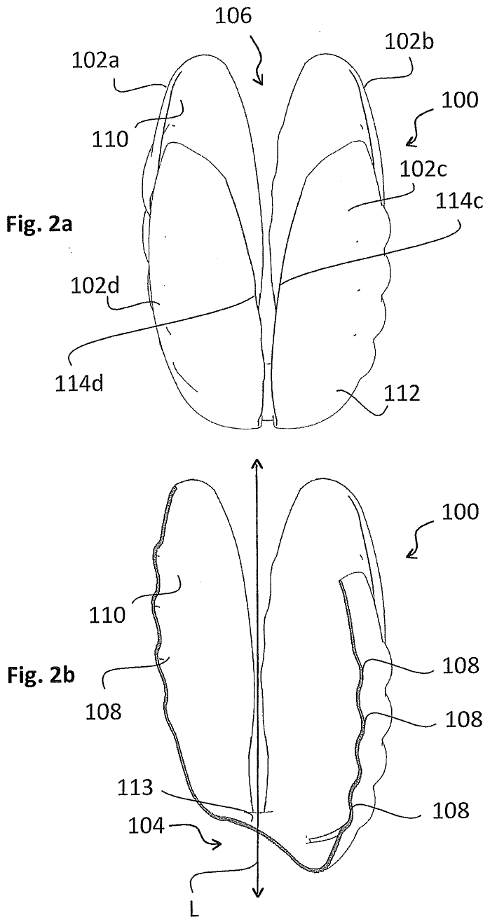

FIGS. 2a, b and 3a, b show a shell in accordance with embodiments herein.

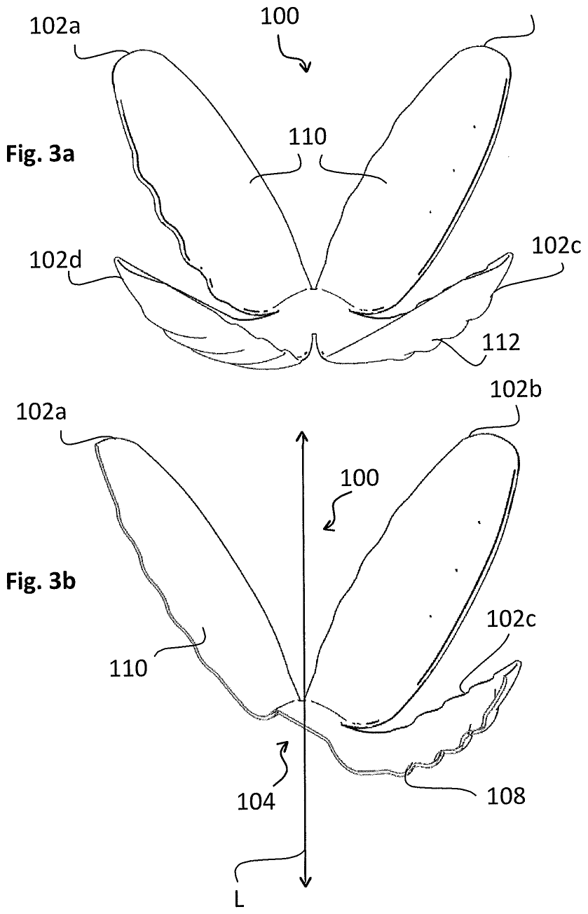

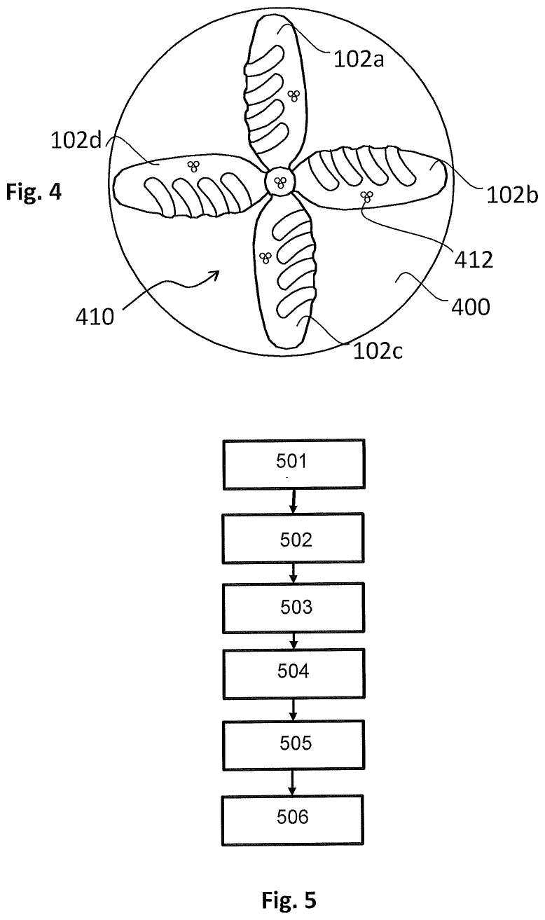

FIG. 4 shows a mold for producing a shell in accordance with embodiments herein.

FIG. 5 is a flow diagram for methods disclosed herein.

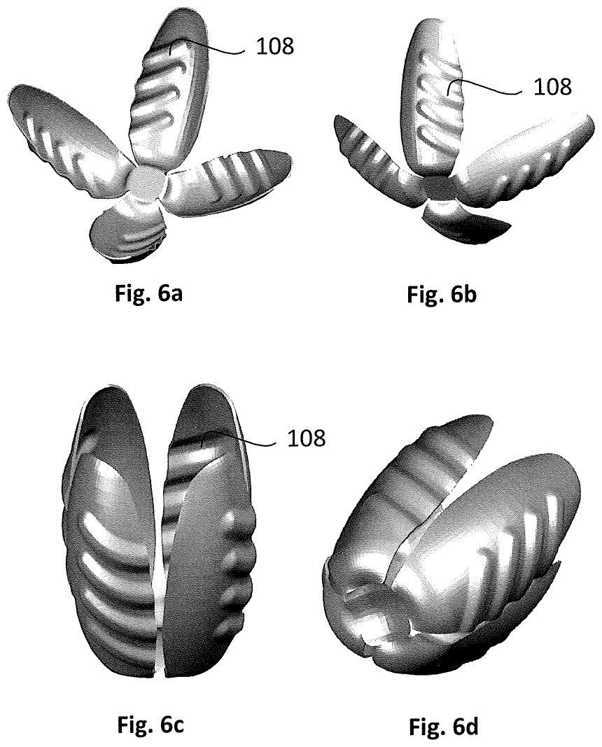

FIG. 6a-d show a shell in accordance with embodiments herein.

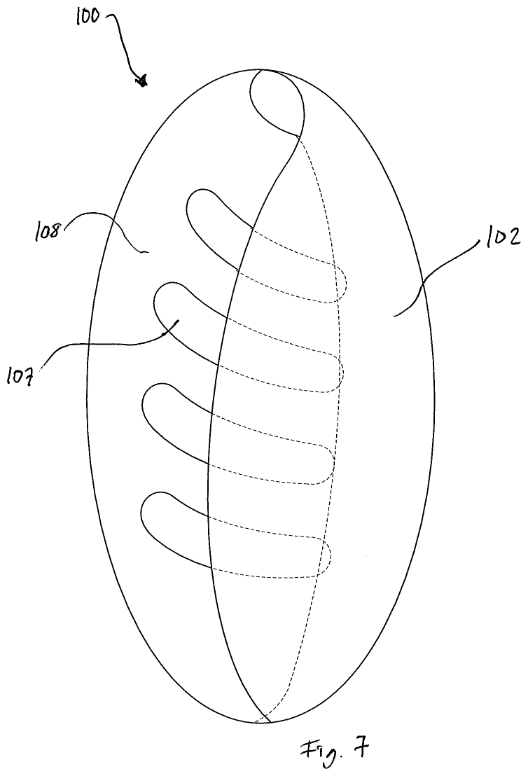

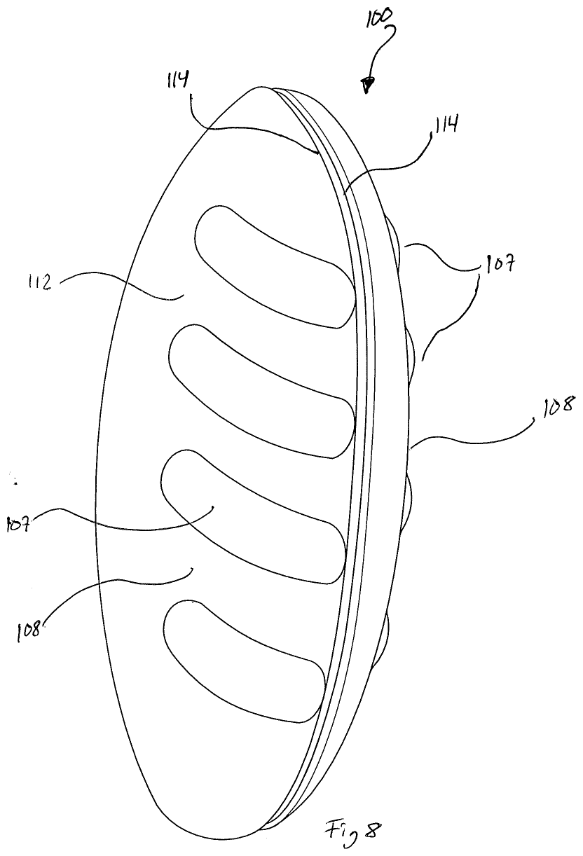

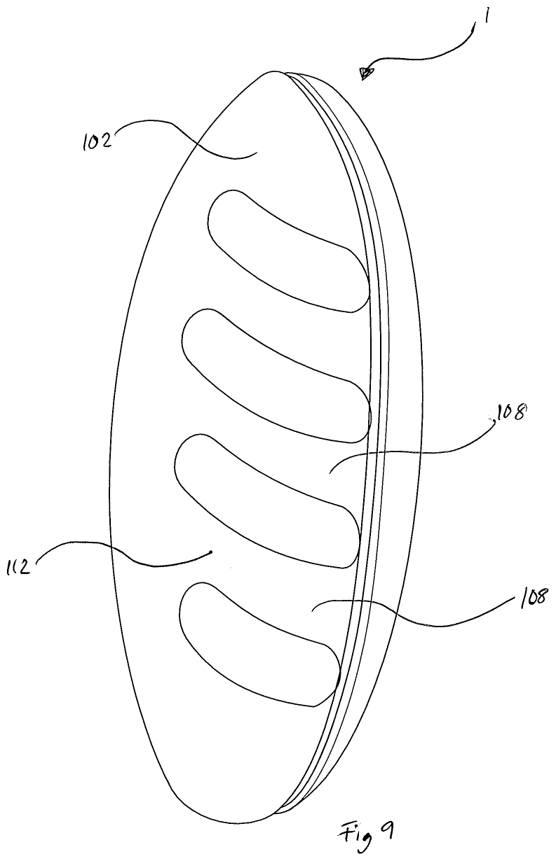

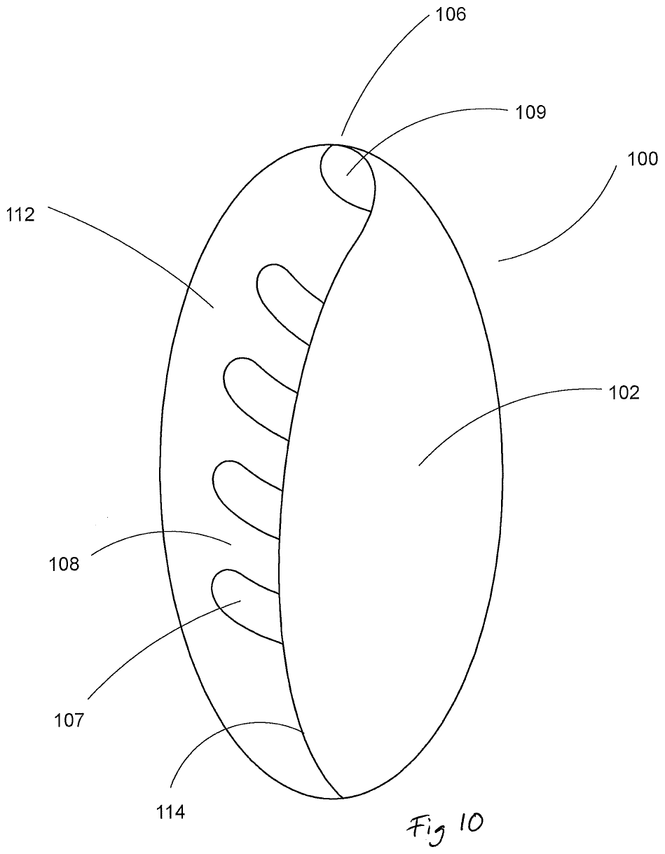

FIG. 7-10 show a shell for use in a catamenial protection device in accordance with embodiments herein.

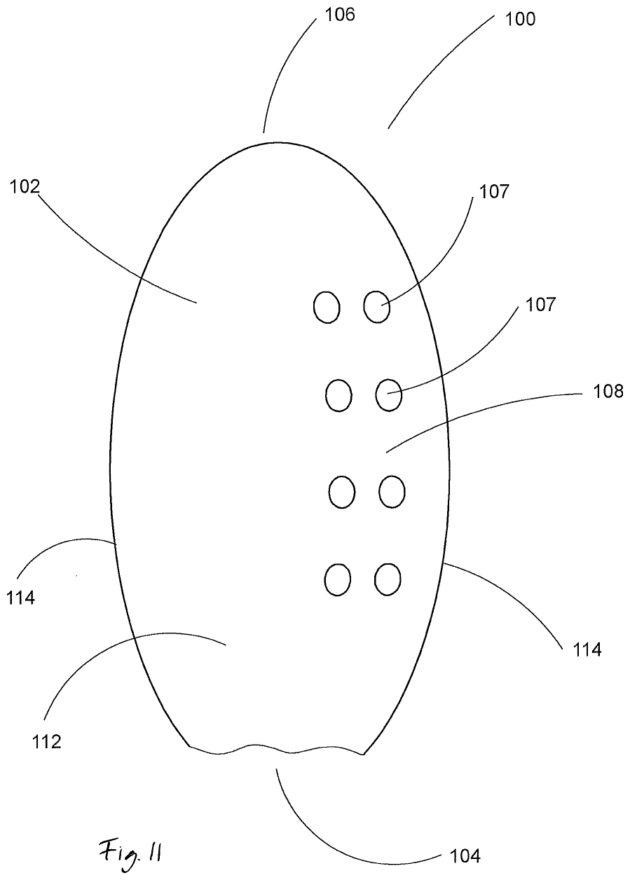

FIG. 11 shows a shell in accordance with embodiments herein.

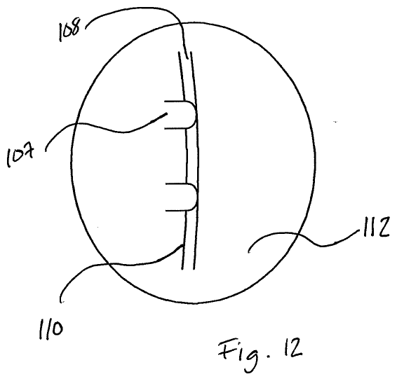

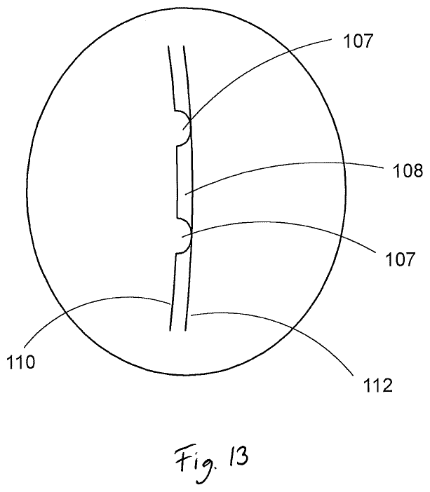

FIGS. 12 and 13 show details of a shell for a catamenial protection device.

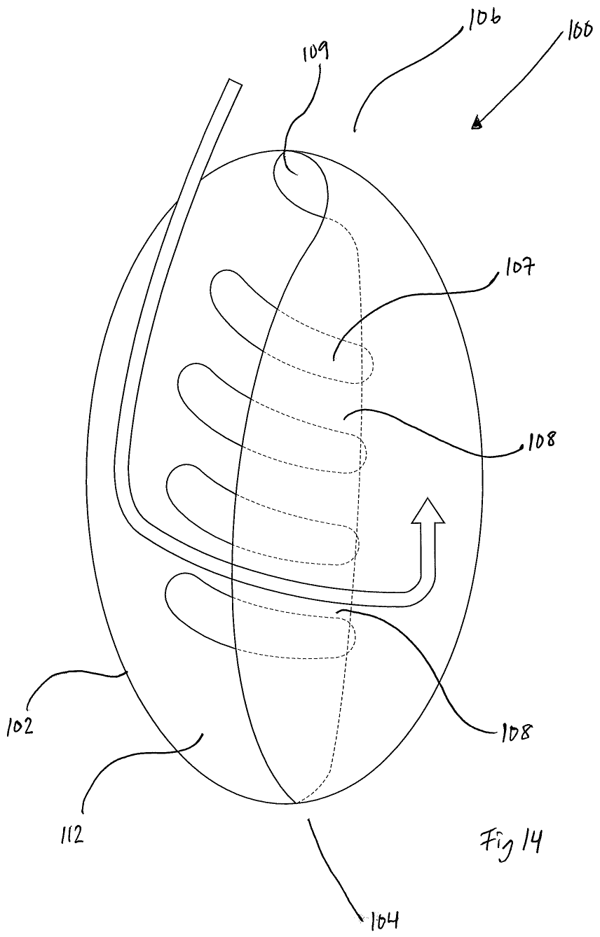

FIG. 14 shows a catamenial protection device in accordance with embodiments herein.

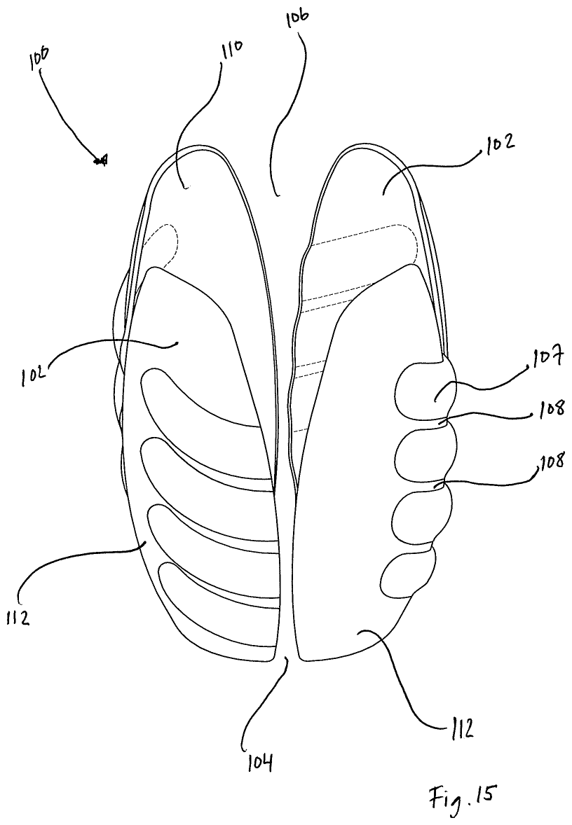

FIG. 15 shows a shell in accordance with embodiments herein.

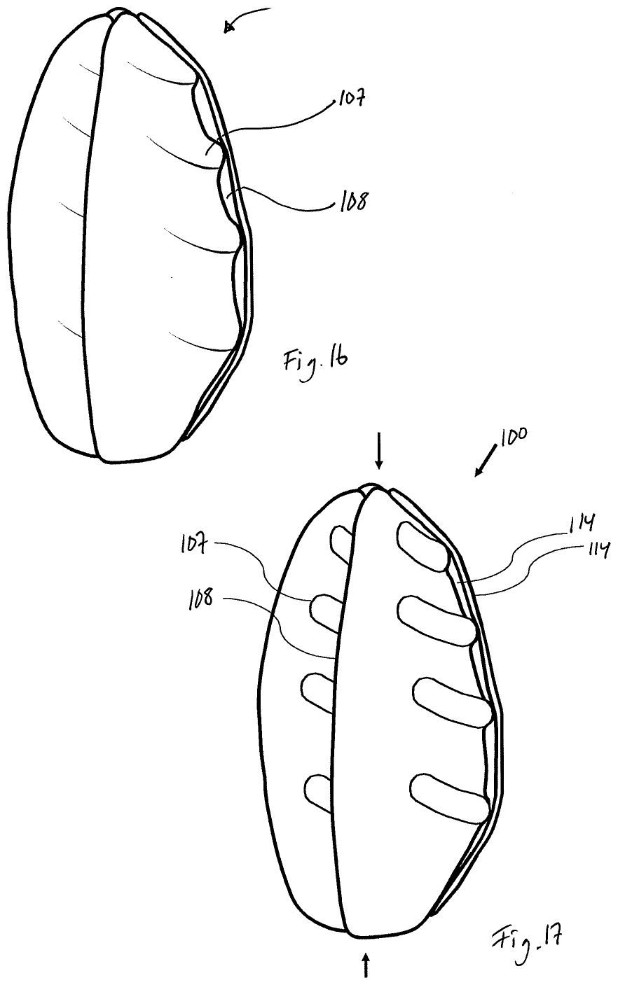

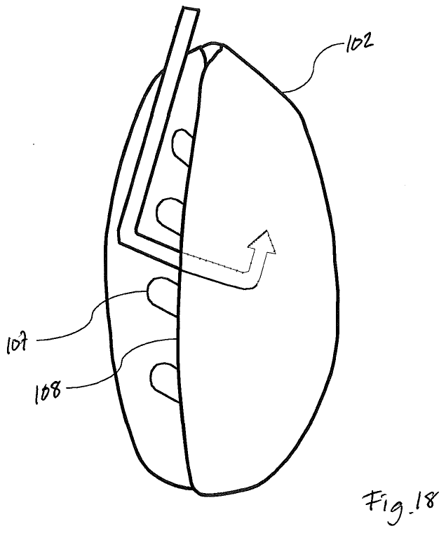

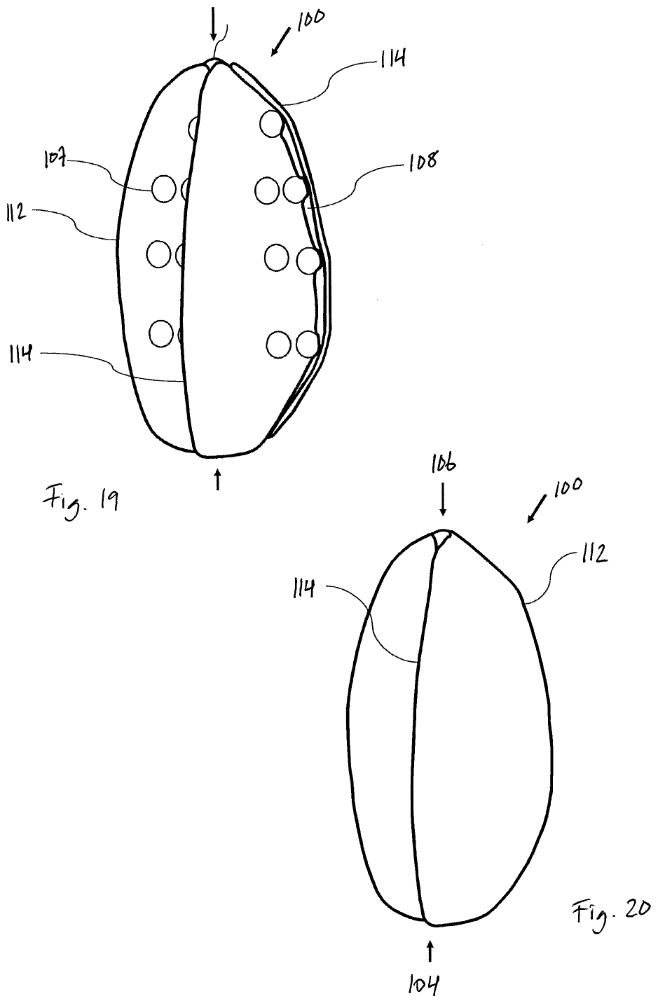

FIG. 16-19 show a catamenial protection device in accordance with embodiments herein.

### DETAILED DESCRIPTION

With reference to the attached drawings, example of embodiment will now be described in detail.

FIGS. 1a and 1b shows a catamenial protection device 1 for absorbing menstrual fluid when positioned inside a vagina in accordance with embodiments herein. The catamenial protection device 1 comprises an inner body 200 comprising material with high fluid absorbing properties. A shell 100 provided with a hard and smooth outer surface 112 is enclosing the inner body 200. The shell 100 comprises at least one elongated portion 102 arranged such that two long sides 114a, 114b of the elongated portion 102 are enabled to at least partly overlap each other. The shell 100 is in this exemplified embodiment provided with four elongated portions 102a, 102b, 102c and 102d. In this exemplified embodiment, the four elongated portions are connected to each other in a first end 104. As may be seen in the figures, the elongated portions are connected to each other via a bottom 113.

The four elongated portions may be folded together whereby the shell 100 will enclose the inner body 200. In FIG. 1, the device 1 is shown in a semi-folded state in order to show how the device is constructed. When ready for use, the elongated portions 102a, 102b, 102c and 102d will be folded together more tightly, totally enclosing the inner body 200. Adjacent long sides 114 of the elongated portions 102 will then be overlapping each other, and the elongated portions 102 may be arranged to enable an opening 109 (not shown) at a second end 106. When the device 1 is in use, the second end will be facing towards a proximal portion of the vagina.

A longitudinal extension of the catamenial protection device is in the FIG. 1b indicated with the axis L. The axis L substantially represents the top-to-bottom extension of any catamenial protection device exemplified herein when the device is in use.

The device 1 may comprise two or more elongated portions 102. The portions may be connected to each other in the first end 104 and are arranged to enable an opening 109 at a second end 106, wherein the second end is facing towards a proximal portion of the vagina when the device 1 is in use. The at least one elongated portion 102 may be arranged such that the overlapping long sides 114a, 114b are enabled to slide in relation to each other, whereby the device 1 is enabled to expand or retract, and whereby the opening 109 at the second end 106 is provided.

The overlapping long sides 114a, 114b are arranged at least partly at a distance from each other whereby one or more paths ways 108 for transporting menstrual fluid are provided between the overlapping long sides 114a, 114b. The distance between the overlapping long sides 114a, 114b serve as an opening in the shell allowing menstrual fluid to flow from the outside towards the centre of the device.

In FIG. 1b an intermediate pouch 300 is provided in which the inner body is placed. The intermediate pouch 300 is thereby placed between the shell 100 and the inner body 200. Also, FIG. 1b discloses a removal means 118 that may for example be a thread, but any other type of elongated form and material that facilitates removal of the menstrual protection device may be used. The removal means may e.g. be a protrusion in the outer casing, a gripping form, a band, a wire, or any other similar means. It is readily understood by the skilled person that any removal means may be used.

The intermediate pouch 300 may comprise perforations sized to allow flow of menstrual fluids. The perforations may allow menstrual fluids to enter into the inner body, but still keep the inner body 200 safe in place. The perforation size may be about 0.05 mm-1 mm, or 0.1-0.5 mm. The intermediate pouch 300 may comprise a membrane allowing fluid to pass into the pouch but restricting fluid from exiting out of the pouch. Such a membrane may be used with or without perforations in the intermediate pouch 300.

The inner body 200 may be placed into an intermediate pouch 300 before the shell 100 is folded around the intermediate pouch 300 and the inner body. By use of such pouch 300, the inner body 200 may be secured inside the pouch 300 before it is arranged in the shell 100.

The shell 100 prevents the absorbing body 200 from getting into contact with the sensitive mucous membrane of the vagina. The distance between the overlapping long sides 114a, 114b may be provided by that the elongated portions 102 is made of a semi-stiff material with a slightly resilient but still persistent material, thus although arranged to enclose the inner body 200 and with the long sides 114a, 114b overlapping each other still providing a distance between the same.

In the first end 104 the elongated portions 102a-d are connected to each other in a bottom 113. The bottom 113 constitutes the lower portion of the folded shell 100 and may serve for attaching a removal means. In a second end 108 the shell 100 forms an opening 109. The opening is formed by that the elongated portions are folded together but not fully closed. Thereby, the overlapping elongated portions 102 will provide the opening 109 in the folded shell 100 allowing for menstrual fluids to flow from the second end 108 and into the inside of the device 1 where it will be absorbed by the inner body 200. It is to be noted that in alternative embodiments, the elongated portions may be fully closed together and the shell 100 is thus provided as a closed shell without any opening in the second end 106. Thus, neither the first end 104 nor the second end 106 is provided with any opening in this exemplified embodiment. In yet another exemplified embodiment, the opening 109 per se may instead in the second end 106 be provided the widest width, or close to the widest width, of the device 1.

It is to be noted that while the device 1 is in use, the elongated portions 102a, 102b, 102c and 102d are enabled to slide against each other in the overlapping areas. Thereby, a tight folded shell 100 may be allowed to slightly open up during use and thus allowing the inner body 200 to expand while absorbing menstrual fluid. Thanks to the overlap of the long sides (shown as 114c and 114d) the shell 100 may expand and still provide for an efficient protection against that the mucous member will get in direct contact with the inner body 200.

Each of the elongated portions 104a, 104b, 104c and 104d may be provided with a slightly concave shape improving the shell 100 to conform to the inner body 200. Further, the number of elongated portions 102 may vary, and the shape of the same will be adapted to provide for a proper fit in relation to the inner body 200. The elongated portions 102a-102d are in embodiments herein shown with a substantially leaf formed shape with two tapering ends and a wider central portion. Each elongated portion 102 is provided with a concave shape with the surface 110 facing inwards, and the convex surface 112 facing outwards.

A catamenial protection device 1 is thus provided that allows for long use throughout a working day. The low absorbing properties of the shell 100 reduces the risk of drying out or irritating the mucous membrane of the vagina. The high absorbing properties of the inner body 200 in combination with the efficient transportation of menstrual fluid from the outside towards the inside of the device ensure both high reliability for long term use as well as a low risk of the growth of bacteria inside the vagina. Thus, the device 1 need not to be replaced during a working day which is especially advantageous under conditions where there is limited access to clean water, soap, toilets, etc. during the working day.

FIGS. 2a-b, 3a-b and 6a-d are different views of a shell 100 for a catamenial protection device 1. In the figures, four elongated portions 102a-d are shown in a semi-folded state. FIG. 2b is a cross sectional view of a shell 100. In a folded state, adjacent long sides 114 of the elongated portions 102 will at least partly overlap each other, but still enable an opening 109 in the end 106 where menstrual fluid may enter the device from the cervix of the vagina. A flow of menstrual fluid serve for that menstrual fluid is passed through from the outside to the inside of the device 1. Thus, the shell 100 provide for that fresh menstrual fluid always will be present on the outer surface being in contact with the sensitive mucous membrane, and old fluid will be passed into the inside of the device 1 where it will be absorbed by the inner body 200. This will decrease the risk of the growth of bacteria on the outer surface which may lead to TSS. Thanks to this, the device 1 can be in use for longer time than traditional tampons without risk of TSS.

FIGS. 4-11 shows a shell 100 in accordance with embodiments herein. The shell 100 in this example comprises one single elongated portion 102 arranged such that two long sides 114a, 114b of the elongated portion 102 are enabled to at least partly overlap each other. In the embodiment shown, the shell 100 comprises only one single elongated portion 102, but it may be understood that the shell may comprise a plurality of elongated portions 102a, 102b, . . . 102x which encloses the inner body 200 (not shown). The elongated portion, or portions, 102 are provided with a shape and form which enable it to enclose to the inner body 200 when folded together.

As further seen in FIG. 7, the single elongated portion 102 is wrapped together to form the shell 100, enclosing the inner body 200 (not shown). The long sides 114a, 114b overlap but are only partly in direct contact with each other thanks to the elongated ridges. Thus, the outer long side abuts the elongated ridges 107. A number of pathways 108 are provided between adjacent ridges 107. The inside of the pathways 108 may be provided with a smooth and hard surface. Thereby the flow of menstrual fluid is further facilitated thanks to low friction. Thus, the relatively low friction surface in the pathways 108 further secure that only fresh menstrual fluid is present on the outside of the device 1.

The single elongated portion 102 may be folded together by more or less rolling it. The form and shape of the elongated portion 102 provide a shape which properly conforms to the inner body when the shell 100 id folded together. The overlapping long sides 114a, 114b are arranged at least partly at a distance from each other whereby one or more paths ways 108 for transporting menstrual fluid are provided between the overlapping long sides 114a, 114b. The pathways 108 transport menstrual fluid from the outside of the device 1 into the inside where it is absorbed by the inner body 200. The shell 100 may however comprise two or more elongated portions 102. The long sides 114 enabled to at least partly overlap each other will thus be a respective long side of two adjacent elongated portions 102.

In an exemplified embodiment, at least one of the long sides 114a may be provided with at least one protruding part 107. The protruding part 107 is arranged to be in contact with the surface of the overlapping long side 114b. Thereby the one or more paths ways 108 for transporting menstrual fluid are provided on one or both sides of the protruding part 107. The pathways 108 is in this embodiment provided by protruding parts 107 shaped like elongated ridges. A plurality of ridges provide for a plurality of pathways 108. As shown in this exemplified embodiment, the ridges 107 and the intermediate pathways 108 between them extend in a substantially transversal direction in relation to the longitudinal axis L of the device. Thus, at least one protruding part 107 with a substantially elongated shape may be extending in a direction arranged with an inclined angle in relation to the axis L. Further, in this embodiment the ridges 107 and thus the pathways 108 are slightly inclined in relation to the axis L. Due to gravity, menstrual fluid will be lead in a downward and inward direction towards the centre of the device 1 and the inner body 200. Thus, a more efficient transport of menstrual fluid will be achieved thanks to the inclination.

The overlapping parts of the long sides 114 may be provided with protruding parts 107 which will serve to ensure that the overlapping parts are kept at a distance from each other. The protruding parts 107 may be provided on either one of the overlapping surfaces, or on both surfaces. The protruding parts may have any suitable shape, still providing for pathways 108 where the menstrual fluid may flow towards the centre of the device 1. The menstrual fluid may flow along the surface of the inner of the elongated portion in the overlapping area. Thereby, the flow of menstrual fluid will flow initially in a more or less spiral formed way towards the inner parts of the device 1.

A plurality of protruding parts 107 may be provided along the long side 114. The protruding parts may have any shape. The protruding parts 107 are exemplified as knobs with a substantially circular shape, or as elongated portions formed in the shape of ribs are provided. The at least one protruding part 107 may thus have a substantially circular shape or a substantially elongated shape, like ribs or ridges.

Menstrual fluid will be transported via the paths ways 108 from the outside of the catamenial protection device 1 towards the inner body 200 which will absorb the menstrual fluid. At least one of the overlapping long sides 114a, 114b may be provided with a plurality of protruding parts 107, whereby the pathways 108 are provided between two adjacent protruding parts.

The overlapping long sides 114a, 114b may be arranged to slide in relation to each other, whereby expansion of the inner body 200 is enabled. The pathways 108 may be arranged to enable transport of menstrual fluid in a substantially transversal direction in relation to the catamenial protection device 1 when the device is in use. The pathways 108 may be provided with a smooth and hard surface 112, whereby transport of menstrual fluid is facilitated. The length of the pathways 108 in the substantially transversal direction may be longer than the thickness of the shell 100.

The shell 100 and/or the inner body 200 may be made of an environmentally friendly material. The environmentally friendly material may be biodegradable may for example be substantially pulp slurry. The pulp or pulp slurry comprises cellulose, hemicellulose and lignin, and may be of a cardboard-like material

The lignin in the shell 100 may be disposed such that the lignin is more evenly distributed on the outer surface than on the inner surface. Thereby, fiber network densification and more bindings between fibers are provided especially close to the surface of the material and a hard and smooth outer surface is provided. The shell 100 may comprise a hot pressed cellulose based material with a densified fiber network outer surface. Thus, the smooth outer surface 112 may be provided by a densified fiber network surface.

Lignin normaly softens at 80-90 degrees. To achieve a more efficient and evenly distribution of the lignin, hot pressing technology with heating temperatures up to 100 or more may be used. but may be even up to 140-150 or even 180-200 or more, especially in the surface of the material. Higher temperatures provides for a hard and smooth surface. The pressing surface, in for example molds, mold cavities, rolls and cylinders, or roll and belt, or roll cavity wall temp may be 150 to 250 degrees or more. Hot pressing using tool forming surface temperature over 110° C., 120° C., 140° C., or 160° C. or even 180° C., 200° C., or 220° C., 250° C. or more may be used.

Smooth metal surfaces provides for smooth surfaces in the resulted product. A metal cavity wall provides for good energy transport to the material and hence an efficient fiber network densification will be achieved.

The at least one elongated portion 102 may be provided with a substantially concave shape conforming to the shape of the inner body 200. The shell 100 and the inner body 200 may comprise environmentally friendly material.

In embodiments herein, the shell 100 comprises a plurality of elongated portions 102 connected to each other in the first end 104 which may be closed. Further, each of the plurality of elongated portions 102 is formed and sized to at least partly enclose the inner body 200.

It is to be noted that the second end 106 directed towards a proximal portion of the vagina should be interpreted as that the second end 106 is to be inserted first into the vagina followed by the first end 104. In practice, when in use the second end 106 of the device 1 will be placed in the vicinity of the cervix.

Since the cervix position and length may differ for different individuals, the position of the second end 106 in relation to the cervix will in practice vary from individual to individual. For some users the second end 106 may be placed in abutment to the cervix and for other users the second end may be placed juxtaposed to the cervix.

The device 1 may be totally environmentally friendly, such as biodegradable, alleviates problems related to waste disposal and thereby reduces negative effects on the environment. Materials of interest may for example be fossil free wood based material.

The shell 100 may be of a material with low fluid absorbing properties which enables the presence of menstrual fluids on the outer surface 112 that is not absorbed into the material. Thereby, the outer surface 112 of the shell 100 will be moist and not dry. Thus, when in use the moist provides low friction between the device 1 and the vaginal wall which serve for comfort and low risk of irritation and chafe.

Environmentally friendly material is to be understood such that the material possibly may be recycled without any substantial environmental effect. Examples of such material is biodegradable material that may be decomposed in nature by micro-organisms such as bacteria or other living organisms.

In the context of embodiments herein, low fluid absorbing properties is to be understood as significantly lower absorbing properties than a high fluid absorbing properties. Thus, a device 1 with the combination of a shell 100 with low fluid absorbing properties together with an inner body 200 with high fluid absorbing properties is especially advantageous as will be explained more in detail.

An example of a material with low fluid absorbing properties in this context is a material that may absorb less water than 20-200% of its own weight, or less than 50% of its own wight, and a high fluid absorbing properties is a material that may absorb more water than 300-1000% of its own weight.

The outside surface 112 of the shell 100 will be in contact with the vaginal walls when the device 1 is in use. The inner surface 110 of the shell 100 will be close to or in contact with the inner body 200, or an intermediate pouch 300 if such pouch is present, when the device 1 is in use. The inner body 200 may be provided with a slightly rounded but substantially cylindrical shape. The protruding part or parts 107 may be dots, circles, dashes, ridges and valleys, or any other protrusions on the shell. The protruding part 107 may be formed on the inside surface 110 of the shell 100, and/or on the outer surface 112 of the shell. The ridges may be longer than the overlap, and may extend even the full width of the elongated portion 102. If ridges are arranged on both overlapping long sides and angled/inclined they may meet crosswise.

The shell 100 and/or the inner body 200 may comprise biodegradable biobased plastics. Examples of such biodegradable plastics are PHA (Polyhydroxyalkanoates), PBS (Polybutylene succinate), Starch blends, Bacterial cellulose, polysaccharides, liquid wood, or materials comprising keratin or casein. Some examples of biobased (but not biodegradable) plastics are PE (polyethene), PET (polyethylene terephthalate), PA (Polyamide), PTT (Polytrimethylene terephthalate) and PLA (polylactide). Fossil but biodegradable plastics such as PBAT (polybutylene adipate terephthalate), and PCL (Polycaprolactone) may also be used. Biodegradable cellulose in the material of the shell may be made from a mix of long fiber kraft, such as NBSK, and short fiber, such as BCTMP. The shell 100 may thus both be capable of being folded without breaking due to presence of the long fibers but will also be provided with a smooth outer surface due to the presence of the short fibers which creates less stress on the mucous membrane of the vagina.

The shell 100 may comprise pulp with several layers comprising different fiber lengths. Short fibers may first be applied for a smooth surface finish, and longer fibers providing for high strength may be applied. Thereby, the outer surface of the shell will be smooth, while the shell 100 still has high strength enable the shell to be folded without breakage.

The material in the shell 100 may have a thickness of about 0.05-2.0 mm, or between 0.1-1.0 mm. In these thicknesses, the shell 100 will be rigid enough to at least partly keep its form, before but also during use. The shell 100 may be formed from a material, such as cardboard, or cardboard-like material cardboard, or material with cardboard-like properties, with high bulk and low density level, for example may such material have a density around 400 kg/m3.

The shell 100 may be of a cardboard-like material. With cardboard-like material is in this context meant that the form stability is relatively hard compared to traditional devices, and that the material typically when deformed to much will result in that the fibers are broken and that the structure thus is deformed permanently.

Elongated parts 102 was tested regarded form stability in dry condition. Test pieces was taken from a shell with four elongated parts 102 each measuring approx. 2.5 cm×5.5 cm. The elongated part was put on a flat force measuring device with edges down and the convex formed outer surface upwards. A force was distributed downwards over approx. 2 cm2 on top of the outer convex surface and was increased until the convex form will give in and the fibers was broked. Measured forces was mostly 5 N to 20 N. A few tests showed as low as 1 N and some over 30 N.

The shell 100 may have a maximal liquid absorption capacity of 0.2-2.0 g H2O per g weight, or not more than 0.5 g H2O per g weight. Hereby, the shell will avoid getting dissolved, and also decrease the exposure to the mucous membrane of the vagina. The term pulp may include materials comprising fibers such as cellulose, minerals and starch, or any combination of the same. The pulp may have a liquid carrier, which may comprise water.

The molding tool may be provided with withdrawal holes, so as to achieve vacuum during use. This will increase the speed and accuracy of the production process. By vacuum is meant that a reduced pressure is applied, i.e. low pressure seen to normal ambient conditions (atmospheric pressure, 1013 mbar).

In embodiments herein, an alternative method of producing the catamenial protection device 1 is provided.

The method comprises providing a molding tool with a molding surface sized and formed as a shell for the catamenial protection device 1. The molding tool may be provided with one single or a plurality of recesses corresponding one or more elongated portions 102 of the shell 100. The elongated portions 102 may be connected in the first end 104 that may be closed.

The method comprises filling the molding tool with a biodegradable plastic, allow the plastic to harden e.g. by letting it cool whereby the shell 100 is formed. Thereafter, the inner body 200 can be arranged inside the shell. The method further comprises folding the plurality of elongated portions 102 towards a second end 106 whereby the shell 100 at least partly encloses the inner body 200. Filling the molding tool with biodegradable plastics may be performed by injecting the biodegradable plastics. Whereby the method will be an injection molding process.

Methods herein may comprise opening the plurality of elongated portions 102 from a closed position to an open position before arranging the inner body 200 inside the shell 100, and subsequently letting the elongated portions 102 return into a closed position at least partly enclosing the inner body 200.

In a folded state, the elongated portions 102a-102d will overlap each other to form a shell enclosing the inner body 200. The longitudinal sides 114 of two adjacent elongated portions 102 will be able to slide in relation to each other, whereby the shell 100 is allowed to expand, or retract. Although two overlapping long sides 114 may partly be in contact with each other, a distance between them is still present providing for pathways 108 allowing menstrual fluid to flow from the outside towards the inside of the device 1 where the fluid will be absorbed by the inner body 200.

Each elongated portion 102 is provided with an inner surface 110 and an outer surface 112. The outer surface 112 is provided with a hard and smooth surface. The hard and smooth outer surface is provided by that a relatively high heating temperature and high pressure is used closest to the outer surface 112 during manufacturing. The hard and smooth surface will provide for low friction against the mucous member during insertion of the device 1, and also for low friction during use. Thereby, the risk of that the device is irritating the mucous member is decreased. Thus, the hard and smooth surface eases insertion of the device and no specific applicator is needed. Further, less material is chafed off. It is to be noted that a high suction catamenial protection device may cause problem for the mucous membrane especially in the presence of small wounds on the mucous. Thus, a shell with a large area with a hard and smooth surface with more of a liquid repelling than high liquid absorbing properties provides for a good relation between the surface of the shell and the mucous member, especially for extended hours of use.

Still further, a hard and smooth surface will facilitate the flow of menstrual fluid due to low friction. In combination with the pathways 108, efficient flow of menstrual fluid will be provided. This does not only result in a more efficient device, it also serves to further secure the presence of fresh fluid thereby minimizing the risk of bacteria growth when the device 1 is in use.

The shell 100 may comprise material of low fluid absorbing properties, whereby transport of menstrual fluid through the shell 100 is provided. In this context, low fluid properties mean that the lion part of surrounding liquids is rest on the surface while a little part is absorbed, while high fluid properties mean that the lion part is absorbed. Thereby, the shell per se will provide for a flow of menstrual fluid from the outside surface 112 right through the material of the shell 100, via the inside surface 110 and further to the inner body 200. Still, the outer surface 112 will be kept smooth, providing for low friction against the mucous member thereby minimizing the risk or irritation. A still low fluid absorbent material in the outer surface 112 will not become dry which if it happens will increase the risk that the surface fastens against the mucous member which may cause irritation and possible damages. The outer surface 112 shall if possible be kept moist but not wet which will help to provide a surface with low friction. Enhancing of the shell surface properties e.g. harder or smoother, might be done by other environmentally friendly material e.g. environmentally friendly polymers or plastics.

The shell 100 may be provided with a rough inner surface 110. An inner surface 110 which is more rough than smooth will provide for the surface 110 to slightly attach to the surface of the inner body 200. Thereby, a more efficient transport of fluid from the inner surface 110 to the inner body 200 will be achieved. The rough inner surface is provided by that a relatively low heating temperature is used closest to the inner surface 110 during manufacturing.

The shell 100 may be made of pulp, for example a mix of long fibre pulp and short fibre pulp. For example, the pulp mix may comprise about 50% bleached long fibre kraft, such as Northern bleached Softwood Kraft and about 50% short fibre pulp, for example aspen bleached chemi-mechanical pulp or BCTMP from hardwood. Long fibres from softwood shows a high properties of bending without breaking, whereby a strong material is provided. Short fibres on the other hand provide for a smooth and even surface with low friction. Thus, in a layered constructure short fibres may be applied first to provide for a smooth and even surface, while longer fibres may be applied after to provide for high strength of a layered product.

The fibres may be solved in water for creating the slurry. A paper chemical may be used as a hydrophobation additive. E.g. an alkyl ketene dimer (AKD) or an ASA glue may be used as a reactive synthetic sizing agent to provide a desired level of hydrophobicity to the shell and to reduce risk of clogging the tool during production.

The inner body 200 comprises material with high fluid absorbent properties. The inner body 200 may comprise a natural material which is easily degradable in nature. For example, the inner body 200 may be made of a wood product such as softwood sulphate treated fluff pulp. The inner body 200 may have an absorption capacity of at least 3 g H20 per g weight, or more than 6 g H20 per g weigh or for example about 8 g H2O per g weight. Further, it may also be possible to have an absorption of up to e.g. 10 g H20 per g weight. The inner body may be a fluff pulp such as e.g. NaturaFluff or similar product.

The inner body 200 may be formed by compression, rolling, folding or in any other way form material into the inner body. In the exemplified embodiments, the inner body 200 is shown with a longer extension along the longitudinal axis L, than the width transversal to the longitudinal axis L. The inner body 200 is shown with a substantially cylindrical form with rounded ends in the top and/or in the bottom.

FIG. 4 is a top view of a molding tool for producing a shell 100 in accordance with embodiments herein. The molding tool 400 comprises one or more molding surfaces 410, in the exemplified embodiment four molding surfaces. Thus, the molding surface shape has a plurality of the elongated portions 102a, 102b, 102c, 102d which are connected at a first closed end 104, being a central portion on the mold.

The molding surface 410 further comprises a number of small withdrawal holes. These holes may be used for exerting a vacuum (under pressure) while the mold it dipped into a slurry, so as to suck the slurry into the mold and form the outer casing. A corresponding mold, which can be heated and has a mating surface may be applied onto the illustrated mold, to dry the pulp or pulp slurry into the shell. The molding surface may further comprise a number of air exhaustion outlets 412 which may be used for exerting air after the shell is formed to release it from the mold. The exhaustion outlets 412 are illustrated as three holes on each of the elongated portions and the central closed end 104.

Further, FIG. 5 shows a flow diagram of a method for producing a catamenial protection device 1 according to embodiments herein. The method comprises providing a molding tool 400 with a molding surface 410 which is sized and formed as an unfolded shell 100 for the catamenial protection device 1. This may be done by for example lowering the mold into a pulp slurry. Further, if a perforated mold is used, having a plurality of withdrawal holes, vacuum (low pressure) may be applied to the withdrawal holes. The molding surface may alternatively be sprayed with slurry.

The protruding elements 107 are in the example provided by forming the elongated elements during a molding process during pressure. The protruding elements may therefore be present on both the outer surface 112 and on the inner surface 110. Alternatively, the protruding elements may be provided solely on the inner surface 110, or the outer surface 120.

Mechanical pressure may be applied through a face of a mating press tool. This tool may be heated so as to heat the pulp or pulp slurry layer and thereby forming the shell 100. The heating temperature may be about 150-300 degrees ° C., whereby the shell 100 is dried in less than 10 seconds, or in less than 5 seconds, or in less than 2 seconds. High temperature in the pressing tool metal being in contact with the cellulose surface provides for reshaping of the surface and provides for increased hydrophobicity in the material. Heating may be performed by heating a first mold half, whereby the heat distribution in the slurry during heating will be warmer closest to the first mold half, and colder closest to the second mold half. It is to be noted that the slurry in contact with the first mold half which is warmer will form the outer, hard and smooth surface of the shell.

Applying mechanical pressure may be repeated a plurality of times. It may be repeated with the same molding and mating tool. It may also be repeated with different molding or mating tools and/or with different distances between the mating tools. It may be possible to perform pressing with a first distance the first time, or times, and to decrease the distance between the mating tool and the molding tool in subsequent pressing cycles. The distance may be decreased by increasing the force with which the mating tool is pressed against the molding tool. It is to be noted that as an alternative to molding, the shell may be produced by starting the pressing/heating procedure from a suitable flat tissue. Still further, a flat tissue later formed may be provided by a producing method establishing pressure and heat by use of rotating cylinders, of which at least one is heated. To extend the pressure time during such a rolling heating pressure process, an extended nip or a steelbelt may be used.

It may further be possible to adapt the molding form of the mating tool in order to compensate for a higher pressure (thus a shorter distance) but keeping e.g. the same curvature of a radii in the shell which is to be molded. Hereby, the same form may be produced despite that the distances between the molding tool and the mating tool is changed (by increasing the force).

As an option, trimming of the shell 100 may be performed. The shell 100 is thus trimmed so that at least a part of the formed shell 100 is removed. Trimming may be conducted after applying mechanical pressure, and/or heating, to the pulp, but before folding the plurality of elongated portions 102.

An inner body 200 is arranged within the shell 100. The inner body 200 may be placed onto the bottom 113 of the shell 100. The inner body 200 may be placed in an intermediate pouch 300 before being placed in the shell 100. Forming of the inner body 200 may be performed with help of a special tool, where the material of the inner body 200 is pressed, rolled, or folded into its final shape.

The plurality of elongated portions 102a, 102b, 102c, 102d is folded towards the second end 106. The elongated portions 102 are folded such that they at least partly enclose the inner body 200 and at the same time provide the opening 109 in the second end 106. The elongated portions 102 may be folded such that long sides 14 of two adjacent elongated portions 102 will overlap each other, in order to form a shell 100. The elongated portions 102 may be pressed towards the inner body 200 to be formed as a tightly fitted catamenial protection device 1.

### EXPERIMENTS, RESULTS AND EFFECTS

### Experiment 1; Long Time Use

6 women tested the product for 12 hours per use.

Result/effect: The women surprisingly found the product not to have
the characteristic odor that usually comes from their earlier use of regular tampons they have worn for longer times, like in this test 12 hours.

The product with inner absorbing part essentially enclosed by a shell with an outer hardened and smoothed surface using hot pressing, a smooth tool surface and a cellulose based material giving a densified fiber network outer surface provides for less odor and hence less bacteria growth.

### Experiment 2; Different Pressing Pressure

The product shell was pressed with different pressure over different areas of the same shell. The pressure provides for the heat to transfer from the pressure tool to the shell due to better contact and also for smoothing the surface using a smooth tool surface. The correct pressure and a temperature over at least 80-90 degress C. provide for the reorganizing of the lignin in the surface and smoothing of the surface. Areas with correct higher pressure according to the invention and areas with not enough pressure where both identified before use. They are also easy identified after use due to the smooth surface achieved by correct pressure according to the invention.

Result/effect: After use it was found that the correct pressurized and smoothed areas showed a surprisingly low or no affinity to the menstrual blood. The areas with lower applied pressure showed blood had become integrated in the shell and the material.

### Experiment 3; Storing of Shell in Cup with Menstrual Blood

The shell, only the shell, was placed in a cup with fresh menstrual blood for 25 hours.

As in experiment 2 the different pressurized areas showed different affinity to menstrual blood. Further in experiment 3 the shell was rinsed in running water. Cup and shell was enclosed in a plastic bag during the 15 hours.

Result/effect: It was found that the correct pressurized areas showed a surprisingly low on no affinity to the menstrual blood. The areas with lower applied pressure showed blood integrated in the shell.

Surprisingly, the white colour of the cellulose material surface having a correct pressurized area was white again after rinsing with water.

As a concluding surprising effect, after rinsing the product with water the areas with hot pressed cellulose-based material with a densified fiber network surface showed almost no or no blood residue in the surface and hence providing for a more vagina and mucous membrane friendly product.

### Experiment 4; Shell with Outer Side “Rough”

A shell was pressed with the outside surface rough and the inside surface smoothened smooth. Hence, the smooth surface is according to the invention; hot pressed and/or heat treated where the lignin is softened and reformed in the surface using raised temperature min 80-90 degrees C. and pressure against a smooth tool or roll surface, but for this experiment arranged on the inner side of the shell, facing the absorbing part. Rough meaning the surface is approx rough like a regular office paper, or slightly rougher, and not hot pressed with a smooth and polished tool surface.

The product was placed in vagina during menstrual period with medium to low blood flow, as a complete product including the absorbing inner body.

After, 6 hours the product was removed.

Result/effect: the testing women reports uncomfort when removing the product with rough outside and that the surface “grips” in the vagina inner walls and/or mucous membrane. Other women expressed the product with rough outside to get stock inside the vagina and being difficult to remove without uncomfort.

Same women reports no such “gripping” when using the product according to the invention with a smooth outside.

### Experiment 5; Shell as Applicator

Due to the cardboard like shell, and its part cylindrical or almost tulip leaves form, the product provides rigidness or stiffness. The product according to the invention was penetrated into vagina.

Result/effect: The cardboard like shell and forms rigidness/stiffness was tested and found helpful during penetration, especially in the beginning and end of a menstrual period when the flow of blood is limited and less.

It was also shown that the shell being longer than the absorbing part and hence protrudes over the absorbing part is advantageous when application/penetration of the product into the vagina. The protruding part of the shell not only covers the absorbing part, but the protruding rounded shell ending also form a tapered ending and when penetrated into the vagina, providing for an easy application and penetration.

### Experiment 6, Removal

During periods with low menstrual fluid or in the beginning or end of a period there is less blood flow. Removal tests were performed in this low fluid period.

Quarter to semi of blood full products according to the invention was removed from vagina during low fluid periods.

Result/effect: The product according to the invention was reported easy to “slide out” spite the low presence of blood, hence there was low friction between the product outer surface and vagina, even with small or no amounts of blood present. This was reported as a significant difference compared with regular tampons which tends stick to the mucous membrane or vagina inner wall, especially in periods of less blood.

### Experiment 7; Alternative Manufacturing Methods

Preliminary data shows a first alternative manufacturing method, the Deep drawing of paper, and also a second alternative manufacturing method the Dry moulding fiber process to shown good result for the manufacturing of the shell as alternative methods as well as combinations of these two and the earlier mentioned, e.g. hot pressing could be dry or wet or one could precede another one.

### Experiment 8; Bacteria Growth

Test performed by 3 women for 2 menstrual periods each.

The inner front part of the vagina was first swabbed for bacteria. Then the product was used for 12 hours in the vagina. Then the product was removed, and the vagina was swabbed a second time again in the same place after use of the product. The swabs were examined by a laboratory at Linköping University hospital.

Result/effect: The laboratory at Linköping University hospital concluded no increase of bacteria from the first swabbing tests, to the second swabbing tests after using the product according to the invention.

The foregoing disclosure is not intended to limit the present invention to the precise embodiment shown in the figures. It is contemplated that various alternate embodiments and/or modifications to the present invention, whether explicitly described or implied herein, are possible in light of the disclosure. Accordingly, the scope of the invention is defined only by the claims.

In a preferred manufacturing method, the elongated portions are manufactured in an open position, spread to each other, to be closed at a later stage, e.g. in assembly and enclosing the inner part. A hinge line is arranged at the first end for folding the cardboard-like elongated portions to fold inwards. The hinge line is in an embodiment with two elongated portions arranged straight across the centre of the first end. In embodiments with three or more elongated portions the hinge line describes a segment of the first end being closer and closer to the elongated portions as the number of elongated portions for the product increases in further alternative embodiments. In further an alternative embodiment the hinge line is arranged at the base of the elongated portion. These hinge line arrangement solves the problem of not being able to bend the cardboard-like elongated portions without permanently damaging the fiber structure of the elongated portions.

The elongated portions are in a preferred embodiment arced along its elongation. The elongated portions are also arced transversal to its elongation. This double arcing and the cardboard-like properties of the elongated portions provides for rigidness of the elongated portions and hence also the rigidness of the product providing for easy penetration and use.

In embodiments, a predetermined hinge line is arranged at the first end, along which the elongated portion is arranged to be folded.

In embodiments, double arced elongated portions have cardboard-like properties.

In embodiments, the shell is moulded in an open position, prepared for a later step of folding the elongated portions inwardly to enclose the inner body.

1: A shell for use in a catamenial protection device for absorbing menstrual fluid when positioned inside a vagina,
wherein said shell is arranged to enclose an inner body of the catamenial protection device, and wherein said shell comprises at least one elongated portion arranged such that the shell is enabled to expand or retract.

2-35. (canceled)

36: A method of producing a shell according to claim 1, the method comprising:
molding a shell by applying a pulp or pulp slurry in a first mold half provided with a molding surface with at least one cavity with a shape corresponding to at least one elongated portion,
pressing said applied pulp or pulp slurry between said first mold half and a mating second mold half,
heating said molded shell at an elevated temperature and under pressure.

37: The method of producing a shell according to claim 36, the method further comprising:
folding said molded shell together whereby the shell is enabled to at least partly enclose an inner body of a catamenial protection device.

38: The method of producing a shell according to claim 37, wherein said at least one cavity is provided with at least one recess with a shape corresponding to at least one protruding part of the shell, and wherein pressing is performed such that said slurry is pressed into said at least one recess.

39: The method of producing a shell according to claim 36, wherein heating is performed by heating said first mold half, whereby the heat distribution in the slurry during heating will be warmer closest to the first mold half, and colder closest to the second mold half.

40: The method of producing a shell according to claim 36, wherein said molding surface is provided with a plurality of cavities with a shape corresponding to a plurality of elongated portions, and an intermediate part providing for molding a bottom in a first end, said bottom is connecting said plurality of elongated portions to each other, and wherein folding the shell is performed by folding said plurality of elongated portions together towards a second end.

41-59. (canceled)

60: A catamenial protection device for absorbing menstrual fluid when positioned inside a vagina,
wherein said catamenial protection device comprises:
an inner body comprising material with high fluid absorbing properties,
a shell provided with a hard outer surface and enclosing said inner body,
wherein said shell comprises at least one elongated portion arranged such that two long sides of the at least one elongated portion are enabled to at least partly overlap each other, and
wherein one or more pathways for transporting menstrual fluid are provided in the overlapping area of said overlapping long sides.

61-89. (canceled)

90: A method of producing a catamenial protection device according to claim 60, the method comprising:
molding a shell by applying a pulp or pulp slurry in a first mold half provided with a molding surface with at least one cavity with a shape corresponding to at least one elongated portion,
pressing said applied pulp or pulp slurry between said first mold half and a mating second mold half,
heating said molded shell at an elevated temperature and under pressure, and
arranging an inner body in the shell.

91: The method of producing a catamenial protection device according to claim 90, the method further comprising:
folding said molded shell together whereby the shell at least partly encloses said inner body.

92: The method of producing a catamenial protection device according to claim 90,
wherein said at least one cavity is provided with at least one recess with a shape corresponding to at least one protruding part of the shell, and wherein pressing is performed such that said slurry is pressed into said at least one recess.

93: The method of producing a catamenial protection device according to claim 90,
wherein heating is performed by heating said first mold half, whereby the heat distribution in the slurry during heating will be warmer closest to the first mold half, and colder closest to the second mold half.

94: The method of producing a catamenial protection device according to claim 90,
wherein said molding surface is provided with a plurality of cavities with a shape corresponding to a plurality of elongated portions, and an intermediate part providing for molding a bottom in a first end, said bottom is connected to said one or more elongated portion, and wherein folding the shell is performed by folding said one or more elongated portion together.
